(19) United States
(12) Patent Application Publication
(10) Pub. No.: US 2026/0141976 A1
(43) Pub. Date: May 21, 2026

(54) WATERMARKING METHOD FOR PROTEIN GENERATIVE MODELS

(71) Applicant: The Trustees of Princeton University, Princeton, NJ (US)

(72) Inventors: Zaixi Zhang, Princeton, NJ (US); Mengdi Wang, Princeton, NJ (US)

(73) Assignee: The Trustees of Princeton University, Princeton, NJ (US)

(21) Appl. No.: 19/389,678

(22) Filed: Nov. 14, 2025

Related U.S. Application Data
(60) Provisional application No. 63/720,954, filed on Nov. 15, 2024.

(51) Int. Cl. G16B 15/20; G16B 40/20; G16B 50/10

(52) U.S. Cl. CPC G16B 15/20; G16B 40/20; G16B 50/10

(57) ABSTRACT

The present disclosure provides a method for embedding watermarks into protein generative models, comprising pretraining an SE(3)-equivariant watermark encoder and decoder, wherein the encoder receives a watermark code and generates a watermark-conditioned structure, and the decoder receives the watermark-conditioned structure and predicts an embedded watermark, and using a watermark-conditioned adaptation to encode a desired watermark code and generate an updated protein generative model by merging the desired watermark code into model weights from an original protein generative model, wherein the protein generative model is fine-tuned with a message retrieval loss and a consistency loss. The watermark-conditioned adaptation includes a gating vector derived from the watermark code. The method enables copyright authentication and tracking of generated protein structures while preserving structural integrity and biological functionality.

### CROSS-REFERENCE TO RELATED APPLICATIONS

This application claims priority to U.S. Application No. 63/720,954, titled “FoldMark: a generalized watermarking method for protein generative models”, filed Nov. 15, 2024, which is hereby incorporated by reference in its entirety.

### TECHNICAL FIELD

The present disclosure relates to watermarking methods for generative artificial intelligence models, and more particularly to a watermarking method for protein generative models that embeds imperceptible identifiers into generated protein structures for copyright authentication and tracking purposes.

### BACKGROUND

Protein structures are fundamental to understanding biological processes and have become central to advances in bioengineering, drug discovery, and molecular biology. The three-dimensional arrangement of amino acids in proteins determines their function, making accurate protein structure prediction and design valuable for developing new therapeutics and understanding disease mechanisms.

Recent developments in generative artificial intelligence have transformed the field of computational protein biology. Machine learning models can now predict protein structures from amino acid sequences with remarkable accuracy, solving challenges that have persisted for decades. These generative models have also enabled the design of novel proteins with desired properties and functions, opening new possibilities for creating custom biological molecules.

The power and accessibility of protein generative models have led to their widespread adoption across research institutions and commercial organizations. These models can generate protein structures for various applications, from basic research to pharmaceutical development. As these technologies become more prevalent, they are being deployed on platforms and made available through application programming interfaces, allowing users to generate protein structures remotely.

However, the increasing availability and capability of protein generative models have raised concerns about intellectual property protection and potential misuse. The ease with which these models can be shared and deployed creates challenges for organizations that invest substantial resources in developing proprietary protein generation technologies. Additionally, the potential for generating harmful biological structures, such as toxins or pathogenic proteins, presents biosecurity considerations that require careful monitoring and control.

Traditional approaches to protecting digital content, such as watermarking techniques used in text and image generation, face unique challenges when applied to protein structures. Protein structures exhibit complex geometric properties and are sensitive to minor modifications that could affect their biological function. The three-dimensional nature of proteins and their inherent symmetries make conventional watermarking methods less effective, as they may disrupt the structural integrity or biological activity of the generated proteins.

Current methods for tracking and authenticating generated content in other domains rely on embedding imperceptible identifiers that can be later extracted for verification purposes. However, adapting these techniques to protein structures requires specialized approaches that account for the unique characteristics of biological molecules, including their flexibility, sensitivity to structural changes, and complex spatial relationships.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for embedding watermarks into protein generative models is provided. The method comprises pretraining an SE(3)-equivariant watermark encoder and decoder, wherein the encoder receives a watermark code and generates a watermark-conditioned structure, and the decoder receives the watermark-conditioned structure and predicts an embedded watermark. The method further comprises using a watermark-conditioned adaptation to encode a desired watermark code and generate an updated protein generative model by merging the desired watermark code into model weights from an original protein generative model, wherein the protein generative model is fine-tuned with a message retrieval loss and a consistency loss.

According to other aspects of the present disclosure, the method may include one or more of the following features. The pretraining may include distorting a structure generated from the watermark encoder. The distorting may include structure cropping, rotation, and noising. The message retrieval loss may be configured to assign different weights to different time steps. The watermark-conditioned adaptation may include a gating vector derived from the watermark code. The fine-tuning may include fine-tuning the watermark decoder to adapt to protein structures specific to a protein domain. The method may further comprise using training-free guidance by adding an additional drift term to a generation process. The method may further comprise using the watermark decoder to generate a decoded watermark from an output of the updated protein generative model. The method may further comprise comparing the decoded watermark to at least one predetermined watermark to determine ownership of the watermark. The method may further comprise receiving the predetermined watermark from a user. The method may further comprise retrieving the predetermined watermark from a database of known watermarks. The method may further comprise receiving the desired watermark code from a user. The method may further comprise generating the original protein generative model. The method may further comprise receiving a selection of a generative application from a library of generative applications to be used to generate the original protein generative model.

According to another aspect of the present disclosure, a non-transitory computer-readable storage device is provided. The non-transitory computer-readable storage device contains instructions that, when executed by one or more processing units, causes the one or more processing units to, collectively, perform the method described above.

According to another aspect of the present disclosure, a system is provided. The system comprises a non-transitory computer-readable storage device as described above and one or more processing units operable coupled to the non-transitory computer-readable storage device.

According to other aspects of the present disclosure, the system may further comprise at least one remote device operably communicating with the one or more processing units, the at least one remote device configured to receive and display information to a user.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1A illustrates a system diagram of a watermarking method for protein generative models.

FIG. 1B illustrates a sequence diagram representing a watermarking and identification process involving multiple parties and components.

FIG. 2 illustrates a pretraining stage of a watermarking system for protein structures.

FIG. 3 illustrates a system diagram of a finetuning stage for embedding watermarks into protein generative models.

FIG. 4 is a table showing metrics for watermarking unconditional protein structure generative models.

FIG. 5 is a table showing metrics for watermarking protein structure prediction models

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

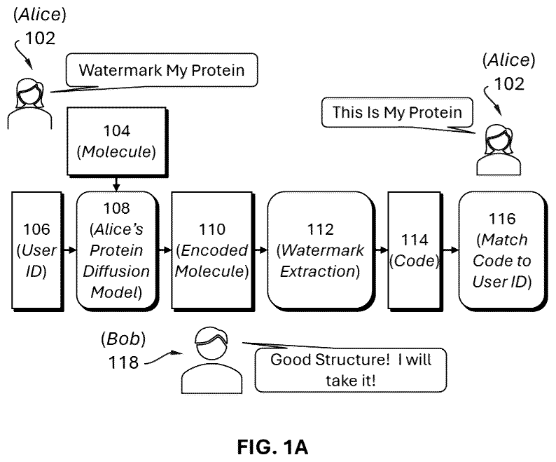

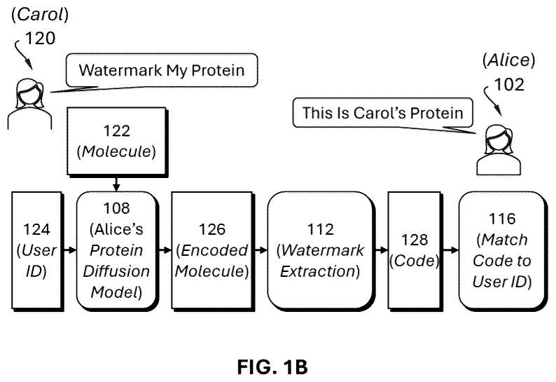

The presently disclosed system, sometimes referred to as FoldMark, provides a watermarking framework for protein generative models that enables copyright authentication and tracking of generated protein structures. The system operates by embedding imperceptible watermark identifiers into protein structures during the generation process. Referring to FIG. 1A, as a simple example, Alice 102, representing a molecule designer and model owner, provides molecule 104 along with a user identifier 106 to a protein diffusion model 108. The protein diffusion model 108 processes these inputs to generate an encoded molecule 110 that contains embedded watermark information. When verification is needed, the encoded molecule 110 can be passed to a module for watermark extraction 112, which may extract the watermark from the encoded molecule 110 to retrieve a code 114. That code can then be processed and compared 116 to the used identifier 106. In this example, this enables Alice 102 to correctly identification herself as the owner of the molecule. Conversely, if Bob 118 finds the encoded molecule, he cannot pass the molecule off as his own. When the encoded molecule is passed for watermark extraction 112, the extracted code will identify Alice 102 as the owner. of unauthorized usage, such as by unauthorized user 118.

Similarly, the system must be robust enough that Alice's diffusion model can encode other people's molecules and ensure they are correctly identified. FIG. 1B demonstrates the practical application of the watermarking system in a multi-user scenario. Carol 120, associated with user ID 124, interacts with Alice's protein diffusion model 108 by providing molecule 122 as input. The protein diffusion model 108 generates encoded molecule 126 containing embedded watermark information specific to Carol's usage. Subsequently, watermark extraction 112 processes the encoded molecule 126 to extract code 128, which is then matched to the user ID 124 through the match code to user ID 116 component. This process enables the system to trace generated protein structures back to specific users, facilitating accountability and ownership verification in protein generation applications.

The presently disclosed system may address the challenges of intellectual property protection in protein generative models while maintaining the structural integrity and biological functionality of the generated proteins.

The method for embedding watermarks into protein generative models may operate through a two-stage approach that balances watermark embedding capabilities with preservation of protein structural quality. The first stage may involve pretraining an SE(3)-equivariant watermark encoder and decoder system. The SE(3)-equivariant architecture may respect the three-dimensional rotational and translational symmetries inherent in protein structures, ensuring that embedded watermarks remain detectable regardless of the spatial orientation of the protein structure.

During the pretraining stage, a watermark encoder may receive a watermark code as input and generate a watermark-conditioned structure. The watermark code may comprise a binary sequence that encodes user-specific information or ownership identifiers. The encoder may learn to embed this watermark information into the protein structure by making subtle adjustments to atomic coordinates while preserving the overall structural integrity of the protein. The watermark-conditioned structure may contain the embedded watermark information in a manner that is imperceptible to standard structural analysis but detectable through the corresponding decoder.

A watermark decoder may receive the watermark-conditioned structure and predict an embedded watermark from the structural data. The decoder may analyze the modified protein structure to extract the embedded watermark code, reconstructing the original binary sequence that was embedded by the encoder. The pretraining process may involve optimizing both the encoder and decoder to achieve high accuracy in watermark embedding and retrieval while minimizing structural distortions to the protein.

The second stage of the method may involve using a watermark-conditioned adaptation to encode a desired watermark code and generate an updated protein generative model. This adaptation process may merge the desired watermark code into model weights from an original protein generative model. The watermark-conditioned adaptation may utilize low-rank adaptation techniques that allow for efficient integration of watermark information without requiring extensive modification of the original model architecture.

The protein generative model may be fine-tuned with two complementary loss functions to achieve the dual objectives of watermark embedding and quality preservation. A message retrieval loss may ensure that embedded watermarks can be accurately extracted from generated protein structures. This loss function may measure the accuracy of watermark recovery by comparing decoded watermarks with the original watermark codes used during generation.

Consistency loss relates to the preservation of the quality of the protein generative model by ensuring that watermarked structures maintain similarity to structures generated without watermarking. The consistency loss may involve comparing outputs from the watermarked model with reference structures from the original model, minimizing deviations that could compromise protein functionality or structural validity. The combination of message retrieval loss and consistency loss may enable the system to generate protein structures that contain embedded watermarks while maintaining the biological and structural properties expected from the original generative model.

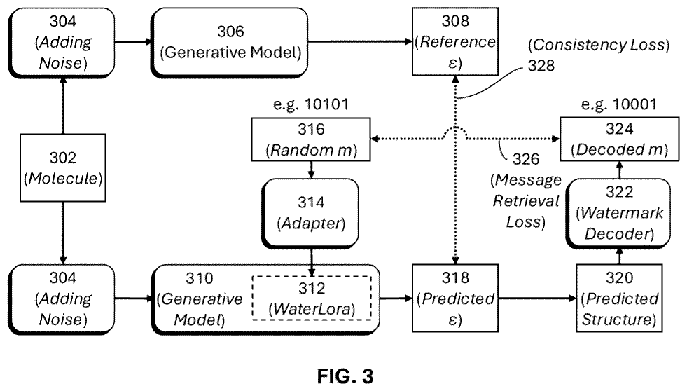

Referring to FIG. 3, the consistency-preserving finetuning stage may implement a dual-pathway architecture that enables simultaneous optimization of watermark embedding and structural quality preservation. A molecule 302 may serve as input to the finetuning process, where the molecule 302 may be processed through parallel pathways to generate both reference and watermarked structures for comparison and optimization.

In the upper pathway of the system, the molecule 302 may be processed through a noise addition module 304 that may add controlled noise to the molecular structure. The noised structure may then be fed to a generative model 306, which may generate a reference structure 308 without watermark embedding. The reference structure 308 may serve as a baseline for comparison during the finetuning process, representing the expected output quality of the original protein generative model.

The lower pathway may process the molecule 302 through a similar noise addition module 304, followed by processing through a generative model 310 that may incorporate watermark embedding capabilities. The generative model 310 may include a watermark adapter 312 that may receive input from an adapter 314. The adapter 314 may process a random watermark code 316, which may comprise a binary sequence such as the example “10101” shown in the figure. The watermark adapter 312 may integrate the watermark information into the generation process, enabling the generative model 310 to produce a predicted structure 318 that may contain embedded watermark information.

The predicted structure 318 may be processed by a watermark decoder 322 to extract a decoded watermark 324. The decoded watermark 324 may comprise a binary sequence that may correspond to the embedded watermark information, such as the example “10001” shown in the figure. The watermark decoder 322 may be fine-tuned during this stage to adapt to protein structures specific to the protein domain, improving the accuracy of watermark extraction from the complex three-dimensional protein structures.

A message retrieval loss 326 may be computed by comparing the decoded watermark 324 with the random watermark code 316. The message retrieval loss 326 may be configured to assign different weights to different time steps during the diffusion process. The weighting scheme may assign larger weights when the time step approaches zero, as generated structures may contain more watermark information at these later stages of the generation process. This time-dependent weighting may enhance the effectiveness of watermark embedding while maintaining generation quality.

A consistency loss 328 may be computed by comparing the reference structure 308 from the upper pathway with a predicted structure 320 from the lower pathway. The consistency loss 328 may ensure that the watermarked structures maintain similarity to the reference structures generated without watermarking. The consistency loss 328 may compare the prediction of the fine-tuned model with the original pretrained model to prevent excessive deviation from original model weights, thereby preserving the biological functionality and structural validity of the generated proteins.

The watermark-conditioned adaptation may include a gating vector derived from the watermark code through a linear transformation process. The gating vector may be computed using a weight matrix and bias vector to modulate Low-Rank Adaptation weight updates. The watermark-conditioned adaptation may utilize Low-Rank Adaptation modules with a rank set to 16 in the default setting to reduce computational costs while maintaining effective watermark embedding capabilities. The gating vector may enable flexible incorporation of watermark information into the model weights without requiring extensive architectural modifications to the original protein generative model.

The method may further comprise using training-free guidance by adding an additional drift term to a generation process. Training-free guidance may provide an alternative approach to watermark embedding that does not require extensive fine-tuning of the protein generative model. The additional drift term may be incorporated into the backward generation process of diffusion models, allowing watermark information to be embedded during structure generation without modifying the underlying model parameters. This approach may enable watermark embedding while preserving the original model architecture and weights.

The method may further comprise using the watermark decoder to generate a decoded watermark from an output of the updated protein generative model. The watermark decoder may process protein structures generated by the updated protein generative model to extract embedded watermark information. The decoded watermark may comprise a binary sequence that corresponds to the watermark code that was embedded during the generation process. The watermark decoder may analyze the three-dimensional coordinates and structural features of the generated protein to reconstruct the embedded watermark information.

The method may further comprise comparing the decoded watermark to at least one predetermined watermark to determine ownership of the watermark. The comparison process may involve matching the decoded watermark against a set of known watermark patterns or codes to establish ownership or origin of the generated protein structure. The predetermined watermark may serve as a reference for verifying the authenticity and source of the generated protein structure. The comparison may utilize similarity metrics or exact matching algorithms to determine correspondence between the decoded watermark and the predetermined watermark.

The method may further comprise receiving the predetermined watermark from a user. A user interface or input mechanism may allow users to specify predetermined watermarks for comparison purposes. The predetermined watermark may be provided in various formats, including binary sequences, alphanumeric codes, or other encoded representations. The user-provided predetermined watermark may be stored and used for subsequent comparison with decoded watermarks extracted from generated protein structures.

The method may further comprise retrieving the predetermined watermark from a database of known watermarks. A database may store a collection of predetermined watermarks associated with different users, organizations, or ownership entities. The retrieval process may involve querying the database using various search criteria or identifiers to locate relevant predetermined watermarks for comparison. The database may maintain associations between watermarks and their corresponding owners, enabling automated identification of protein structure origins.

The method may further comprise receiving the desired watermark code from a user. The desired watermark code may be specified by a user through an input interface or configuration system. The desired watermark code may comprise a binary sequence, alphanumeric string, or other encoded format that represents user-specific information or ownership identifiers. The user-provided desired watermark code may be processed and embedded into protein structures during the generation process.

The method may further comprise generating the original protein generative model. The original protein generative model may be created through training on protein structure datasets using various machine learning architectures. The generation process may involve training diffusion models, flow-based models, or other generative architectures on protein structural data. The original protein generative model may be configured to generate protein structures from input sequences or other conditioning information before watermark embedding capabilities are added.

The method may further comprise receiving a selection of a generative application from a library of generative applications to be used to generate the original protein generative model. A library of generative applications may contain various pre-implemented protein generative model architectures and configurations. The selection process may allow users to choose from different generative approaches, including diffusion-based models, flow-based models, or transformer-based architectures. The selected generative application may serve as the foundation for creating the original protein generative model before watermark adaptation.

The watermarking system may support user identification applications where unique watermarks are assigned to each user, enabling traceability back to specific users through watermark database comparison. In user identification scenarios, each user may be assigned a distinct watermark code that serves as a unique identifier. When protein structures are generated, the embedded watermarks may be extracted and compared against a database of user-specific watermarks to determine the identity of the user who generated the structure. This traceability mechanism may enable accountability and tracking of protein generation activities across multiple users.

The method may include detection applications for copyright protection where successful watermark extraction serves as proof of rightful ownership and indicates artificial generation. In copyright detection scenarios, the presence of a valid watermark in a protein structure may serve as evidence that the structure was generated using a specific watermarked model. The extraction of a recognizable watermark may distinguish artificially generated protein structures from naturally occurring or experimentally determined structures. This detection capability may provide legal and technical evidence for copyright enforcement and intellectual property protection.

The system may achieve nearly 100% bit accuracy in recovering watermarks from encoded protein structures when the watermark code is less than 16 bits. The high accuracy performance may be maintained for watermark codes ranging from 4 bits to 16 bits in length. The bit accuracy may represent the percentage of correctly recovered bits in the decoded watermark compared to the original embedded watermark code. This performance level may enable reliable watermark detection and user identification for practical applications.

The watermark recovery accuracy may decline as the length of the watermark code increases beyond 16 bits, affecting the reliability of the embedded watermark. Watermark codes longer than 16 bits may experience reduced recovery accuracy due to the increased complexity of embedding longer sequences in protein structures without compromising structural integrity. The accuracy degradation may result from the limited capacity of protein structures to accommodate extensive watermark information while maintaining biological functionality. Advanced encoding techniques and optimization algorithms may be developed to embed longer watermark codes without compromising recovery accuracy.

The method may be tested on specific protein structure prediction models including ESMFold and MultiFlow, and de novo structure design models like FrameDiff and FoldFlow. The testing may demonstrate the generalizability of the watermarking approach across different types of protein generative models. ESMFold and MultiFlow may represent protein structure prediction models that generate structures from amino acid sequences. FrameDiff and FoldFlow may represent de novo structure design models that generate novel protein structures with desired properties. The successful application across these diverse model types may validate the versatility of the watermarking framework.

The watermarking framework may exhibit robustness against various post-processing techniques including structure cropping, rotation, translation, and coordinate noising. Structure cropping may involve removing portions of the protein structure while maintaining the embedded watermark in the remaining segments. Rotation and translation operations may test the SE(3)-equivariant properties of the watermark encoder and decoder. Coordinate noising may involve adding random perturbations to atomic coordinates to simulate experimental uncertainties or structural modifications. The robustness against these post-processing techniques may ensure watermark detectability under realistic usage scenarios.

The system may demonstrate resistance to adaptive attacks including fine-tuning attacks and multi-message attacks that attempt to erase or obscure the original watermark. Fine-tuning attacks may involve retraining the watermarked model using clean protein data to remove watermark embedding capabilities. Multi-message attacks may attempt to inject additional watermarks to cover or interfere with the original embedded watermarks. The resistance to these adaptive attacks may be achieved through integrated design approaches and data augmentation strategies during training. The robustness against adaptive attacks may ensure the reliability of watermark detection even when malicious users attempt to circumvent the protection mechanisms.

A non-transitory computer-readable storage device may contain instructions that, when executed by one or more processing units, cause the one or more processing units to collectively perform the method for embedding watermarks into protein generative models. The non-transitory computer-readable storage device may comprise various forms of persistent storage media including solid-state drives, hard disk drives, optical storage media, or other computer-readable storage technologies. The storage device may store executable code, data structures, and configuration parameters necessary for implementing the watermarking method.

The instructions stored on the non-transitory computer-readable storage device may include program code for pretraining the SE(3)-equivariant watermark encoder and decoder. The instructions may further include code for implementing the watermark-conditioned adaptation process that merges desired watermark codes into model weights from original protein generative models. The stored instructions may encompass the algorithms and procedures for fine-tuning protein generative models with message retrieval loss and consistency loss functions.

The instructions may include code modules for processing watermark codes, generating watermark-conditioned structures, and extracting embedded watermarks from protein structures. The storage device may contain libraries and frameworks for implementing SE(3)-equivariant neural network architectures, diffusion model processing, and Low-Rank Adaptation techniques. The instructions may further include code for implementing training-free guidance mechanisms and drift term calculations for the generation process.

A system may comprise the non-transitory computer-readable storage device and one or more processing units operably coupled to the non-transitory computer-readable storage device. The one or more processing units may include central processing units, graphics processing units, tensor processing units, or other specialized computing hardware capable of executing machine learning algorithms and neural network computations. The processing units may be configured to access and execute the instructions stored on the non-transitory computer-readable storage device to perform the watermarking method.

The one or more processing units may be operably coupled to the non-transitory computer-readable storage device through various connection interfaces including SATA connections, PCIe interfaces, USB connections, or network-based storage connections. The coupling may enable the processing units to read instructions and data from the storage device and write results and intermediate computations back to the storage device. The processing units may include sufficient memory and computational resources to handle the complex calculations involved in protein structure generation and watermark embedding.

The system may be configured to distribute computational tasks across multiple processing units to accelerate the watermarking process. The processing units may operate in parallel to handle different aspects of the watermarking method, including encoder training, decoder optimization, and generative model fine-tuning. The system may include memory management capabilities to efficiently handle large protein structure datasets and model parameters during processing.

The system may further comprise at least one remote device operably communicating with the one or more processing units. The at least one remote device may be configured to receive and display information to a user. The remote device may include user interface components such as displays, keyboards, mice, touchscreens, or other input and output mechanisms that enable user interaction with the watermarking system. The remote device may comprise desktop computers, laptops, tablets, smartphones, or other computing devices capable of network communication.

The at least one remote device may communicate with the one or more processing units through various communication protocols and network connections including Ethernet, Wi-Fi, cellular networks, or other wired or wireless communication technologies. The communication may enable users to submit protein structures for watermarking, specify watermark codes, configure system parameters, and receive results from the watermarking process. The remote device may provide a user interface for monitoring the progress of watermarking operations and accessing generated protein structures.

The remote device may be configured to display watermarking results, including decoded watermarks, ownership verification information, and structural quality metrics. The display capabilities may include visualization of protein structures, watermark embedding statistics, and comparison results between original and watermarked structures. The remote device may provide interactive features that allow users to explore generated protein structures and verify watermark embedding success.

The system may support multiple remote devices simultaneously, enabling multi-user access to the watermarking capabilities. Each remote device may be associated with specific user credentials and watermark codes, allowing the system to track and manage watermarking operations across different users. The remote devices may receive personalized information and results based on their associated user identities and access permissions.

### Example

### Watermarking State-of-the-Art Protein Generative Models

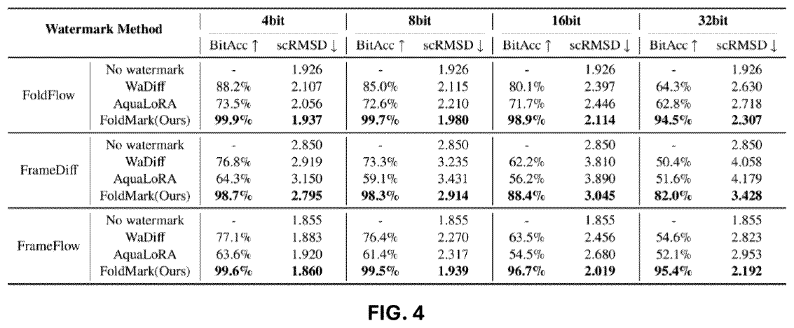

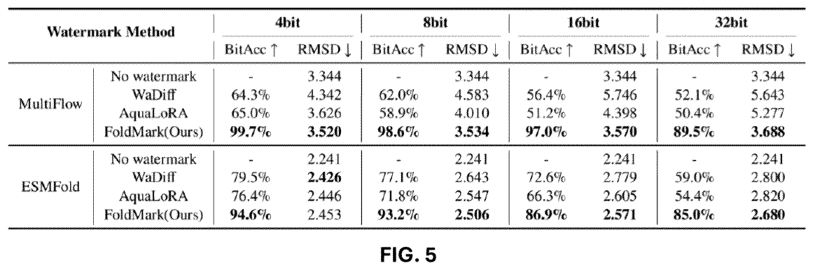

In FIGS. 4 and 5, experiments of watermarking unconditional protein structure generative models (i.e., FoldFlow, FrameDiff, and FrameFlow) and protein structure prediction models (i.e., MultiFlow, and ESMFold) are shown. The watermark code length was varied from 4 to 32 and the bit prediction accuracy (BitAcc) and the structural validity (scRMSD and RMSD) were measured. To benchmark the performance of FoldMark, two watermark methods from the image domain, WaDiff and AquaLoRA were also adapted, for comparison. Generally, the performance degrades with the increase of watermarking capacity, i.e., more watermark bits. On most cases with less than 16 bits, FoldMark achieves nearly 100% bit accuracy on watermark code recovery from encoded protein structures with minimal influence on structural validity (measured by scRMSD and RMSD). Therefore, FoldMark is a generalized and effective method for protein generative model protection.

### Applications in Detection and User Identification

As discussed with respect to FIGS. 1A and 1B, this example shows two applications of FoldMark. The scenario involves Alice, the model owner responsible for training, releasing the pretrained model, and deploying the inference code on the platform. Bob, a thief, downloads Alice's model and code to generate protein structures, falsely claiming ownership of the copyrights. Carol registers as a user on the server and utilizes the API to generate protein structures. In the detection, the successful extraction of a watermark from structures serves as proof of Alice's rightful ownership of the copyright and indicates that the structure is artificially generated. In user identification, Alice assigns a unique watermark to each user. By extracting the watermark from generated structures, it becomes possible to trace it back to Carol by comparing it with the watermark database and regarding the most similar user id. Traceability goes beyond detection, enabling copyright protection for different users by identifying the source of infringement.

In Table 1, the identification accuracy for different generative models is shown with different numbers of users. While FoldMark achieves strong performance with small groups of users, it becomes much more challenging for identification among a larger number of users (e.g., 10⁶).

TABLE 1
Performance of FoldMark user identification accuracy.

| Model | 10³ users | 10⁴ users | 10⁵ users | 10⁶ users |
|---|---|---|---|---|
| FoldFlow | 0.970 | 0.970 | 0.943 | 0.900 |
| FrameDiff | 0.705 | 0.393 | 0.309 | 0.225 |
| FrameFlow | 1.000 | 0.992 | 0.980 | 0.931 |
| MultiFlow | 1.000 | 0.996 | 0.940 | 0.817 |
| ESMFold | 0.903 | 0.824 | 0.450 | 0.334 |

### Robustness Against Post-Processing and Adaptive Attacks

In real applications, the malicious user may take post-processing or design adaptive attacks to bypass the safeguarding of FoldMark. Here, three common post-processing methods for the protein structure and two adaptive attacks were considered in Table 2. Adaptive attacks involve fine-tuning the watermarked model using clean protein data to erase the watermark, or performing a multi-message attack, where additional watermarks are injected to obscure the original ones. We can observe that FoldMark is robust to cropping, translation, and rotation because the watermark information is encoded into each residue and the watermark decoder is SE(3) invariant. Due to the integrated design and data augmentation, FoldMark is resistant to finetuning and multi-message injection.

TABLE 2
Performance of FoldMark under post-processing and adaptive attacks. Protein
post-processing include structure cropping (keeping 50% of the whole sequence),
randomly translating & rotating the whole structure, and adding Gaussian noise
to the coordinates (strength 0.2). Adaptive attacks include fine-tuning the
watermarked model with clean protein data to erase the watermarking capability
and multi-message attack that try to inject additional watermarks to cover
the original ones. We conduct experiments on the 16 bits setting.

| | No | | | | | Multi- |
|---|---|---|---|---|---|---|
| Model | Attack | Cropping | Trans&Rotate | Noising | Finetune | Msg |
| FoldFlow | 0.989 | 0.961 | 0.990 | 0.910 | 0.920 | 0.947 |
| FrameDiff | 0.884 | 0.860 | 0.882 | 0.793 | 0.769 | 0.860 |
| FrameFlow | 0.967 | 0.906 | 0.960 | 0.871 | 0.870 | 0.948 |
| MultiFlow | 0.970 | 0.864 | 0.972 | 0.826 | 0.924 | 0.950 |
| ESMFold | 0.869 | 0.829 | 0.874 | 0.805 | 0.856 | 0.862 |

### Methods

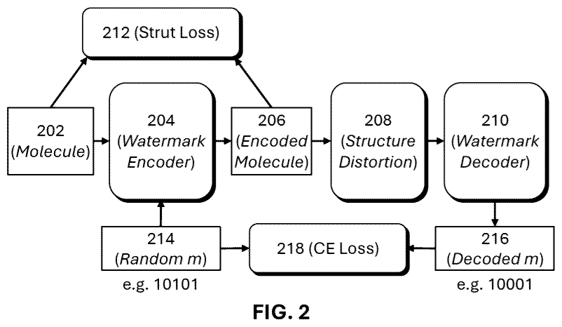

FIGS. 2 and 3 provide an overview of the method used in the example. Inspired by previous works, FoldMark consists of two main stages: Watermark Encoder/Decoder Pretraining and Consistency-Preserving Finetuning. The pretraining stage enables the watermark encoder and decoder to learn how to embed watermark information into the structure space and accurately extract it. The finetuning stage equips pretrained protein generative models with watermarking capabilities while preserving their original generative performance (Consistency-preserving). FoldMark is a versatile method that can be applied to various mainstream protein structure generative models. A diffusion-based model is used as an example and the details of FoldMark are presented below.

### Watermark Encoder/Decoder Pretraining

A watermark encoder [symbol] and decoder [symbol] are first trained such that [symbol] can correctly retrieve the watermark message m embedded by ′ [symbol] .

$${ℒ}_{Pretrain} = {x , m , f}_{} [ {ℒ}_{BCE} ( 𝒟 ( f ( 𝒲 ( x , m ) ) )$$

where x represents the protein structure data and m denotes the string of binary watermark code. γ>0 is a hyperparameter to control the strength of structure adjustment for watermarking. f represents a randomly selected structure distortion as data augmentation. The pool of data augmentation includes random rotation/translation, adding Gaussian noise to protein coordinates, and randomly cropping the protein structure. [symbol] _{BCE}( [symbol] (f( [symbol] (x,m) and ∥ [symbol] (x,m) correspond to the CE Loss and Struct Loss in FIG. 2 respectively.

### Consistency-Preserving Finetuning

Instead of finetuning all the parameters of the generative model, part of the protein generative model was selectively fine-tuned with LoRA and the watermark decoder as shown in FIG. 3. The other parameters including the watermark embedder [symbol] and the reference model are kept unchanged. Details of watermark module are discussed in the next subsection.

Here the diffusion-based protein generative model (e.g., FrameDiff and RFDiffusion) were taken as an example to construct the fine-tuning loss. The diffusion model typically involves two critical components known as the forward and backward process, where the forward process gradually noises the original protein structure x₀ into xₜ for t∈{1, . . . ,T} and the model learns to predict the original structure ϵ_{θ}(xₜ) based on xₜ. There are two losses in the fine-tuning: the consistency loss for regularization and the message retrieval loss to encourage correct watermark retrieval. In the consistency loss [symbol] _{c}, the prediction of the fine-tuned model is compared with the original pretrained model so that the finetuned model weights will not deviate too much from the original ones. For the watermark retrieval loss [symbol] ₘ, one can take a single reverse step with respect to xₜ to obtain

$${{x}^{‵}}_{t} = ( {x}_{t} - \sqrt{1 - {\overline{α}}_{t}} {ϵ}_{θ} ( {x}_{t} ) ) / \sqrt{{\overline{α}}_{t}} ,$$

and then feed it into the decoder to predict the watermark code. In sum, both optimization objectives above were incorporate and the consistency-preserving finetuning loss was formulated as:

$${ℒ}_{Finetunc} = {x , t , m}_{} [ {ℒ}_{c} ( {ϵ}_{θ} ( {{x}^{‵}}_{t} ) , {ϵ}_{{θ}_{ref}} ( {x}_{t} ) ) + η \cdot \frac{t - T}{T} {ℒ}_{m} ( 𝒟 ( {{x}^{‵}}_{t} ) , m ) ]$$

where η controls the trade-off between consistency loss [symbol] _{c} and watermark retrieval loss [symbol] ₘ. An additional weight

$$\frac{t - T}{T}$$

for the retrieval ross was placed because the generated structure contains more information of watermark as t→0 and better performance was observed in experiments.

### Watermark-Conditioned LoRA

Inspired by previous works in image domains (e.g., AquaLoRA and EW-LoRA), watermark-conditioned LoRA was used to save the computation costs of fine-tuning and flexibly embed watermark information in the generation process. The computation formula for Watermark-conditioned LoRA in FoldMark can be expressed as:

$$Δ W ( m )$$

where A∈ [symbol] and B∈ [symbol] are the low-rank matrices, and G∈ [symbol] is the gating vector derived from the watermark code. The operator ⊙ denotes element-wise multiplication, where G modulates the rows of A×B. This formulation maintains efficiency while allowing flexible incorporation of watermark information.

To input the watermark information into the fine-tuned model, we utilize an adapter layer that converts a watermark code of length l into a gating vector G. Specifically, the watermark code m={b₀, b₁, . . . , bₗ} is passed through a linear transformation defined as:

$$G ( m )$$

where W_{g}∈ [symbol] is the linear transformation matrix, and b_{g}∈ [symbol] is the bias vector. Here, bᵢ∈{0,1} represents the binary state of the i-th bit in the watermark code. The gating vector G modulates the LoRA weight updates by scaling the rows of the low-rank update A×B.

During the generation process, when embedding a watermark into the model, the gating vector G was computed based on the watermark code. The resulting LoRA weight update ΔW is added to the original model weights to produce the watermarked model weights:

$${W}_{watermarked} = W + αΔ W ( m )$$

where α is a scaling factor controlling the impact of the watermark on the model weights. FoldMark applies LoRA to all linear and attention layers in structural prediction modules. In contrast, AquaLoRA applies LoRA to linear and convolutional layers in U-Net.

### Difference Between FoldMark and Baseline Methods

The two methods most similar to FoldMark are the baseline approaches, WaDiff and AquaLoRA. The differences, however, include but are not limited to the following points:
(i) FoldMark leverage state-of-the-art SE(3)-equivariant graph transformer as WaterMark Encoder and Decoder. Due to the intrinsic combination with convolutional neural networks (U-Net), CNNs are leveraged as encoder/decoder for WaDiff and AquaLoRA, which limits their performance in the protein domain.
(ii) In FoldMark, customized data augmentation strategies are proposed (e.g., structure cropping, rotation, noising) for robust training. In contrast, the data augmentation strategies in image domains can hardly transfer to protein structures.
(iii) As protein structures are flexible and sensitive, therefore more difficult for watermark retrieval, a customized loss function for consistency-preserving fine-tuning is proposed. The message retrieval loss properly assigns different weights to different time steps, helping keep the generation quality while explicitly enhancing watermark retrieval success rates (larger weights when t→0). In contrast, AquaLoRA only uses consistency-preserving losses and performs not well in watermark retrieval.
(iv) For watermark-conditioned LoRA, FoldMark employs a gating vector derived from the watermark code, ensuring independence from the rank choice in LoRA. In contrast, AquaLoRA integrates a diagonal matrix into the LoRA structure, often requiring large ranks (e.g., 320) to embed the watermark information, thereby incurring substantially higher parameter overhead.

Regarding the fine-tuning strategy, FoldMark takes an additional step by fine-tuning the watermark decoder to adapt to the intricate protein structures specific to the protein domain. In contrast, AquaLoRA keeps the decoder fixed, which results in suboptimal watermark retrieval performance.

Comparison with Other Watermarking Methods

Traditional watermarking techniques developed for Large Language Models (LLMs) and diffusion models are not directly transferable to protein structure data due to the distinct and complex characteristics of protein structures. Protein structures exhibit flexibility, sensitivity, and geometric intricacy, requiring specialized methods for embedding and retrieving watermarks without compromising data integrity or model performance.

Similar methods, such as WaDiff and AquaLoRA, embed watermarks into the U-Net backbone of Stable Diffusion models for image generation. While effective in the image domain, these approaches face significant challenges in protein generative models. The use of convolutional neural networks (CNNs) as encoder-decoder components in WaDiff and AquaLoRA limits their performance in the protein domain, as CNNs are not inherently designed to handle the spatial and rotational properties of protein structures.

FoldMark overcomes these limitations by leveraging state-of-the-art SE(3)-equivariant graph transformers for both the Watermark Encoder and Decoder, ensuring geometric consistency and superior performance. Additionally, FoldMark introduces customized data augmentation strategies, such as structure cropping, rotation, and noising, to enhance the robustness of training. These strategies are tailored to protein structures and are not directly transferable from image-based methods.

To address the challenges of protein flexibility and sensitivity, FoldMark incorporates a novel consistency-preserving loss function for fine-tuning, with message retrieval loss assigning dynamic weights to different time steps (e.g., larger weights as t→0). This approach balances the preservation of generation quality with explicit improvements in watermark retrieval success rates. In contrast, AquaLoRA relies solely on standard consistency-preserving losses, resulting in suboptimal performance for protein watermarking.

Furthermore, FoldMark employs a gating vector derived from the watermark code for watermark-conditioned Low-Rank Adaptation (LoRA), ensuring independence from rank selection. This design avoids the parameter overhead associated with AquaLoRA's reliance on diagonal matrix modifications, which require large ranks (e.g., 320) to embed watermark information effectively.

Finally, FoldMark incorporates an additional fine-tuning step for the watermark decoder, allowing it to adapt to the intricate protein structures. This targeted optimization significantly improves watermark retrieval performance compared to AquaLoRA, which keeps its decoder fixed, leading to limited adaptability and reduced effectiveness.

In summary, FoldMark addresses the unique challenges of protein generative models by combining advanced architectural design, robust training strategies, and innovative fine-tuning approaches, achieving superior performance in protecting protein generative models compared to existing methods.

### Experimental Settings

Datasets. Watermark encoders/decoders were trained, and protein generative models fine-tuned, using the monomers from the PDB dataset, focusing on proteins ranging in length from 60 to 512 residues with a resolution better than 5° A. This initial dataset consisted of 23,913 proteins. Following previous work, data was refined by applying an additional filter to include only proteins with high secondary structure content. For each monomer, DSSP was used to analyze secondary structures, excluding those with over 50% loops. This filtering process resulted in 20,312 proteins.

Implementations. The FoldMark model is pretrained for 20 epochs and fine-tuned for 10 epochs with Adam optimizer, where the learning rate is 0.0001, and the max batch size is 64. The batching strategy from FrameDiff was used of combining proteins with the same length into the same batch to remove extraneous padding. In the LoRA, the rank is set as 16 in the default setting. γ and η are set as 2. The results corresponding to the checkpoint with the best validation loss were reported. It takes less than 48 hours to finish the whole training process on 1 Tesla A100 GPU.

Baselines. FoldMark is the first watermarking method specifically designed for protein structure generative models. For comparison, two state-of-the-art watermarking methods originally developed for image generation were adapted: WaDiff and AquaLoRA. Both baseline models were designed for image diffusion models, such as Stable Diffusion. Since most protein generative models are also diffusion-based, the recommended hyperparameters from the original works were applied.

### DISCUSSION

In this example, the study demonstrates the feasibility of embedding watermarks into protein generative models and their outputs through our proposed method, FoldMark. This two-stage approach successfully preserves the quality of protein structures while embedding user-specific information for copyright authentication and tracking. Extensive experiments on various protein structure prediction and design models confirm the effectiveness and robustness of FoldMark against post-processing and adaptive attacks, with minimal impact on the original structure quality. This provides a potential solution for addressing ethical concerns, such as copyright protection, in the application of generative AI to protein design.

A number of implementations have been described. Nevertheless, it will be understood that various modifications may be made without departing from the spirit and scope of the disclosure. Accordingly, other implementations are within the scope of the following claims.

What is claimed is:

1. A method for embedding watermarks into protein generative models, comprising:
pretraining an SE(3)-equivariant watermark encoder and decoder, wherein the encoder receives a watermark code and generates a watermark-conditioned structure, and the decoder receives the watermark-conditioned structure and predicts an embedded watermark; and
using a watermark-conditioned adaptation to encode a desired watermark code and generate an updated protein generative model by merging the desired watermark code into model weights from an original protein generative model, wherein the protein generative model is fine-tuned with a message retrieval loss and a consistency loss.

2. The method of claim 1, wherein the pretraining includes distorting a structure generated from the watermark encoder.

3. The method of claim 2, wherein the distorting includes structure cropping, rotation, and noising.

4. The method of claim 1, wherein the message retrieval loss is configured to assign different weights to different time steps.

5. The method of claim 1, wherein the watermark-conditioned adaptation includes a gating vector derived from the watermark code.

6. The method of claim 1, wherein the fine-tuning includes fine-tuning the watermark decoder to adapt to protein structures specific to a protein domain.

7. The method of claim 1, further comprising using training-free guidance by adding an additional drift term to a generation process.

8. The method of claim 1, further comprising using the watermark decoder to generate a decoded watermark from an output of the updated protein generative model.

9. The method of claim 8, further comprising comparing the decoded watermark to at least one predetermined watermark to determine ownership of the watermark.

10. The method of claim 9, further comprising receiving the predetermined watermark from a user.

11. The method of claim 9, further comprising retrieving the predetermined watermark from a database of known watermarks.

12. The method of claim 1, further comprising receiving the desired watermark code from a user.

13. The method of claim 1, further comprising generating the original protein generative model.

14. The method of claim 13, further comprising receiving a selection of a generative application from a library of generative applications to be used to generate the original protein generative model.

15. A non-transitory computer-readable storage device containing instructions that, when executed by one or more processing units, causes the one or more processing units to, collectively, perform a method of claim 1.

16. A system comprising:
a non-transitory computer-readable storage device of claim 15; and
one or more processing units operable coupled to the non-transitory computer-readable storage device.

17. The system of claim 15, further comprising at least one remote device operably communicating with the one or more processing units, the at least one remote device configured to receive and display information to a user.
